(19) United States
(12) Patent Application Publication
(10) Pub. No.: US 2005/0100936 A1
(43) Pub. Date: May 12, 2005

(54) Methods and compositions related to prediction of drug response

(75) Inventors: Verena Lutz, Muenchen (DE); Friedemann Krause, Penzberg (DE); Manuela Poignee, Dietramszell/Schoenegg (DE); Thomas Walter, Penzberg (DE); Baerbel Porstmann, Zunzgen (CH); Martin Werner, Freiburg (DE); Silke Lassmann, Freiburg (DE)

Correspondence Address: ROCHE MOLECULAR SYSTEMS INC;PATENT LAW DEPARTMENT, 1145 ATLANTIC AVENUE, ALAMEDA, CA, 94501, US

(73) Assignee: ROCHE MOLECULAR SYSTEMS, INC., Alameda, CA (US)

(21) Appl. No.: 10/927,669

(22) Filed: Aug. 26, 2004

(30) Foreign Application Priority Data
Aug. 28, 2003 (EP) 03018783.5

(51) Int. Cl. C12Q 1/68

(52) U.S. Cl. 435/6

(57) ABSTRACT

The present invention is directed to a method for determining whether a patient suffering from cancer will be responsive to a treatment with a 5-Fluoro-Uracil and/or 5-Fluoro-Uracil analogs. Specifically, the invention relates to methods of determining the expression levels of certain mRNAs to determine responsiveness to drug treatment.

### FIELD OF THE INVENTION

The present invention relates to the field of prediction of responsiveness to drug cancer treatment using biomarker analysis. In particular, the present invention relates to the field of prediction of susceptability of patients suffering from colorectal cancer for 5-FU and/or 5-FU analogs.

### BACKGROUND

Colorectal cancer (CRC), which remains one of the major death causing cancers in the western world, can be cured in about one-third of patients by surgical resection of the primary tumor alone. However, the remaining patients will already have developed occult or distant metastasis at the time of clinical manifestation of the primary tumor and will receive adjuvant (post-surgery) chemo- and/or radiotherapy. Together with the patient's clinical data, histopathological classification of the tumor and staging according to the TNM categories define the criteria for adjuvant therapy.

As the current standard, nodal negative tumors (T1-4, N0, M0) are treated by complete resection of the primary tumor alone, whereas cases with histopathological evidence of lymph node involvement (T1-4, N1-2, M0) will receive chemo- and/or radiotherapy after surgical removal of the primary tumor. Despite a constant improvement of diagnostic tools and relevant markers as well as treatment strategies [Ragnhammar, P., et al., Acta Oncol. 40 (2001) 282-308], prognosis and/or response prediction to adjuvant therapy is still an unsolved issue [Iqbal, S., and Lenz, H. J., Curr. Oncol. Rep. 3 (2001) 102-108; Kumar, S. K., and Goldberg, R. M., Curr. Oncol. Rep. 3 (2001) 94-101].

The enzymes thymidine phosphorylase (TP), dihydropyrimidine dehydrogenase (DPD) and thymidylate synthase (TS) are involved in the metabolism of pyrimidines, and hence eventually involved in proliferation of normal as well as pathologically transformed cells. TS is the central and limiting enzyme for de novo synthesis of pyrimidines and therefore also for RNA/DNA synthesis, DPD is involved in the degradation of uracil and thymine to inactive waste products and TP controls intracellular thymidine levels [Diasio, R. B., and Johnson, M. R., Pharmacology 61 (2000) 199-203]. Moreover, TP was shown to be identical to the molecule platelet-derived endothelial cell growth factor (PD-ECGF) [Furukawa, T., et al., Nature 356 (1992) 668; Sumizawa, T., et al., J. Biochem. 114 (1993) 9-14] and exhibits angiogenic properties in a number of solid tumors [Takebayashi, Y., et al., J. Natl. Cancer Inst. 88 (1996) 1110-1117; Griffiths, L, and Stratford, I. J., Br. J. Cancer 76 (1997) 689-693]. These properties may render TP, DPD and/or TS expression valuable prognostic and chemopredictive markers in CRC.

Furthermore, due to their involvement in pyrimidine metabolism, all three enzymes are also important for the efficacy of 5-FU and 5-FU related chemotherapeutic agents [Diasio, R. B., and Johnson, M. R., Pharmacology 61 (2000) 199-203; WO 02/44423]. Whereas TS is the main target of such chemotherapeutics, the enzymes TP and DPD function in the activation and degradation of these agents and their intermediates, respectively. As such, the three enzymes, individually or the TS/DPD and TP/DPD ratios, have been implicated as markers for prognosis and/or response prediction to adjuvant chemotherapy in CRC [Metzger, R., et al., Clin. Cancer Res. 4 (1998) 2371-2376; Salonga, D., et al., Clin. Cancer Res. 6 (2000) 1322-1327; Takenoue, T., et al., Ann. Surg. Oncol. 7 (2000) 193-198; Araki, Y., et al., Kurume Med. J. 48 (2001) 93-98; Edler, D., et al., J. Clin. Oncol. 20 (2002) 1721-1728; Kornmann, M., et al., J. Gastrointest. Surg. 6 (2002) 331-337]. In fact, high TS levels have been linked to the resistance of tumors to 5-FU chemotherapy [Copur, S., et al., Biochem. Pharmacol. 49 (1995) 1419-1426; Wang, W., et al., Cancer Res. 61 (2001) 5505-5510; Johnston, P. G., et al., Cancer Res. 55 (1995) 1407-1412; Bathe, O. F., et al., Cancer J. Sci. Am. 5 (1999) 34-40] and low DPD expression has been correlated to severe toxicity to 5-FU chemotherapeutic agents [Wei; X., et al., J. Clin. Invest. 98 (1996) 610-615, Johnson, M. R., et al., Clin. Cancer Res. 5 (1999) 2006-2011]. The wide range of TS and DPD expression and the associated effects on chemotherapy outcome appear to be, at least in part, due to a polymorphism in the TS promotor enhancer region [Marsh, S., et al., Int. J. Oncol. 19 (2001) 383-386; lacopetta, B., et al., Br. J. Cancer 85 (2001) 827-830] and a common point mutation within intron 14 of the DPD gene [van Kuilenburg, A. B., et al., Clin. Cancer Res. 6 (2000) 4705-4712; Raida, M., et al., Clin. Cancer Res. 7 (2001) 2832-2839], respectively. Despite a number of studies examining TP, DPD and TS expression in CRC, the role of the enzymes is still conflicting. This is mainly due to diverse study protocols, analysing TP, DPD and TS expression with respect to either 1) mRNA, protein or enzyme activity, 2) in primary tumors or distant metastases and 3) patients treated with various protocols of neo- or adjuvant radio-/chemotherapy.

In view of the outlined prior art, the problem to be solved was the identification of those parameters, which of all potential parameters suggested in the prior art actually are indicative for responsiveness to 5-FU and/or 5-FU analogs and to determine, under which conditions these parameter(s) provide for the highest possible specificity.

### SUMMARY OF THE INVENTION

The present invention is directed to a method for determining whether a patient suffering from cancer is susceptable to a treatment with 5-Fluoro-Uracil or a 5-Fluoro-Uracil analogs comprising:
a) determinating a value for Thymidine Phosphorylase (TP) mRNA expression in a clinical sample;
b) determinating a value for Dihydropyrimidine Dehydrogenase (DPD) mRNA expression in said clinical sample;
c) determinating a ratio of the value obtained in step a) and the value obtained in step b); and
d) determinating whether the ratio obtained in step c exceeds a predetermined cut off value.

In order to avoid false positve results, it is advantageous if the cut off value for the TP/DPD ratio is at least 3 or at least 3.7. On the other hand, in order to avoid false negative results, it is advantageous if the cut off value for the TP/DPD ratio is not higher than 10 or not higher than 8.2.

In a particular embodiment, there is an additional determination of the absolute or relative expression level of Dihydropyrimidine Dehydrogenase and level of expression below a certain cut off value is additionally indicative for responsiveness to 5 Fluoro-Uracil or a respective analogs.

The inventive method is particularly useful for determing responsiveness of patients suffering from colorectal cancer.

### DETAILED DESCRIPTION OF THE INVENTION

In the study underlying the invention, TP, DPD and TS mRNA expression was examined in a unique group of 102 patients with CRC, using microdissected, formalin-fixed and paraffin-embedded primary tumor samples for quantitative RT-PCR (QRT-PCR) in the LightCycler® system. The correlation of TP, DPD and TS mRNA expression and the TS/DPD and TP/DPD ratios to tumor histology as well as to patient prognosis and response prediction to 5-FU adjuvant chemotherapy was examined.

Thymidine phosphorylase (TP), dihydropyrimidine dehydrogenase (DPD) and thymidilate synthase (TS) mRNA expression in CRC was examined with emphasis on their value as prognostic markers in general and as predictive markers for 5-FU chemotherapy. A method of TP, DPD and TS mRNA quantification using RT-PCR and the LightCycler® system (Roche Applied Science) was developed and applied to microdissected formalin-fixed paraffin-embedded tumor tissues of 102 cases of CRC. In contrast to other studies [Johnston, P. G., et al., Cancer Res. 55 (1995) 1407-1412] [Metzger, R., et al., Clin. Cancer Res. 4 (1998) 2371-2376; Salonga, D., et al., Clin. Cancer Res. 6 (2000) 1322-1327], this study retrospectively examined TP, DPD and TS mRNA expression in primary tumors and evaluated the progostic impact and clinical response of patients to 5-FU chemotherapy by correlation of enzyme levels to patient follow up visits. Although TP, DPD and/or TS expression have been analysed in primary CRC tumors with respect to prognosis [Takebayashi, Y., et al., J. Natl. Cancer Inst. 88 (1996) 1110-1117; Araki, Y., et al., Kurume Med. J. 48 (2001) 93-98; Edler, D., et al., J. Clin. Oncol. 20 (2002) 1721-1728; Sanguedolce, R., et al., Anticancer Res. 18 (1998) 1515-1520; Paradiso, A., et al., Br. J. Cancer 82 (2000) 560-567; Findlay, M. P., et al., Br. J. Cancer 75 (1997) 903-909; Allegra, C. J., et al., J. Clin. Oncol. 20 (2002) 1735-1743], this study examined the prognostic and predictive value of TP, DPD and TS mRNA expression in a group of CRC cases of unique sample size, treatment sub-groups, follow-up data and standardised tissue sampling and preservation.

Detailed analysis of TP, DPD and TS mRNA expression in 102 cases of CRC revealed a wide range of expression levels for all three enzymes. Similar studies have been performed [Iqbal, S., and Lenz, H. J., Curr. Oncol. Rep. 3 (2001) 102-108; Metzger, R., et al., Clin. Cancer Res. 4 (1998) 2371-2376; Mori, K., et al., Int. J. Oncol. 17 (2000) 33-38]. These results underline the concept of intertumor heterogeneity.

Finally, the association of TP and TS mRNA levels with a particular tumor histopathology were also reflected by the TP/DPD and TS/DPD ratios. One explanation for these findings may relate to the biological functions of these enzymes. TP, also known as platelet-derived endothelial cell growth factor [Furukawa, T., et al., Nature 356 (1992) 668; Sumizawa, T., et al., J. Biochem. 114 (1993) 9-14], is associated with angiogenesis in a number of solid tumors [Takebayashi, Y., et al., J. Natl. Cancer Inst. 88 (1996) 1110-1117; Griffiths, L, and Stratford, I. J., Br. J. Cancer 76 (1997) 689-693], and TS may be regarded as a marker of metabolic activity and cellular proliferation [Pestalozzi, B. C., et al., Br. J. Cancer 71 (1995) 1151-1157; Backus, H. H., et al., J. Clin. Pathol. 55 (2002) 206-211; Pestalozzi, B. C., et al., Br. J. Cancer 71 (1995) 1151-1157]. Higher TP and TS mRNA expression in “early” tumors may reflect their activity in promoting vascular support and tumor cell proliferation, which is reduced upon progression to favour, for example, tumor cell invasion and metastasis. In fact, low levels of TP, DPD and TS mRNA expression levels have been associated with a favourable response to 5-FU chemotherapy [Metzger, R., et al., Clin. Cancer Res. 4 (1998) 2371-2376; Salonga, D., et al., Clin. Cancer Res. 6 (2000) 1322-1327]. From this study's data, it is exactly this group of tumors with progressed UICC stages, i.e. those patients receiving adjuvant chemotherapy, which express lower TP and TS mRNA levels.

Previous reports have discussed TP, DPD and TS mRNA expression, alone or in combination, as potential markers for prognosis [Takenoue, T., et al., Ann. Surg. Oncol. 7 (2000) 193-198; Edler, D., et al., J. Clin. Oncol. 20 (2002) 1721-1728; Sanguedolce, R., et al., Anticancer Res. 18 (1998) 1515-1520; Paradiso, A., et al., Br. J. Cancer 82 (2000) 560-567; Allegra, C. J., et al., J. Clin. Oncol. 20 (2002) 1735-1743] and/or response prediction for 5-FU chemotherapy in CRC [Metzger, R., et al., Clin. Cancer Res. 4 (1998) 2371-2376; Salonga, D., et al., Clin. Cancer Res. 6 (2000) 1322-1327; Araki, Y., et al., Kurume Med. J. 48 (2001) 93-98; Johnston, P. G., et al., Cancer Res. 55 (1995) 1407-1412]. In addition, the TP/DPD and/or TS/DPD ratios have recently been implicated as prognostic and/or predictive markers [Kornmann, M., et al., J. Gastrointest. Surg. 6 (2002) 331-337; Ishikawa, T., et al., Cancer Res. 58 (1998) 685-690]. The present study identified the TS/DPD ratio as a potential prognostic marker, with higher TS/DPD ratios correlating with poorer overall survival in CRC patients receiving resection alone.

More importantly, the present study revealed a significant correlation of DPD mRNA levels and the TP/DPD ratio with disease-free survival after 5-FU chemotherapy, whereby low DPD mRNA levels and a high TP/DPD ratio predict a longer disease-free survival. This may be related to the fact that low DPD levels alone or low DPD levels and high TP levels (high TP/DPD ratio) will stabilize the active level of 5-FU. In contrast, neither TP, DPD or TS mRNA levels or the TP/DPD or TS/DPD ratio had any predictive value for overall survival.

Whereas previous studies have addressed TP, DPD or TS protein expression in primary CRC tumors by immunohistochemistry [Takebayashi, Y., et al., J. Natl. Cancer Inst. 88 (1996) 1110-1117; Edler, D., et al., J. Clin. Oncol. 20 (2002) 1721-1728; Paradiso, A., et al., Br. J. Cancer 82 (2000) 560-567; Findlay, M. P., et al., Br. J. Cancer 75 (1997) 903-909; Allegra, C. J., et al., J. Clin. Oncol. 20 (2002) 1735-1743], protein content [Sanguedolce, R., et al., Anticancer Res. 18 (1998) 1515-1520] or enzyme activity [Araki, Y., et al., Kurume Med. J. 48 (2001) 93-98], this study employed TP, DPD and TS mRNA analysis by quantitative RT-PCR, as this method is easy to standardize and works well for large series of formalin fixed, paraffin embedded, microdissected tissue samples. Determining any prognostic or predictive value of these enzymes on the RNA level may be complicated by post-transcriptional, fixation related (and associated functional) modifications [Kawakami, K., et al., Clin. Cancer Res. 7 (2001) 4096-4101].

In order to validate the study's approach, microdissected cells from control tissues were initially screened. The results did reflect previously published data, with, for example, high TP levels in inflammatory cells [Fox, S. B., et al., J. Pathol. 176 (1995) 183-190] and high DPD levels in the liver [Guimbaud, R., et al., Cancer Chemother. Pharmacol. 45 (2000) 477-482; Johnston, S. J., et al., Clin. Cancer Res. 5 (1999) 2566-2570]. Furthermore, tumors were reported to have lower DPD levels [Johnston, S. J., et al., Clin. Cancer Res. 5 (1999) 2566-2570; Miyamoto, S., et al., Int. J. Oncol. 18 (2001) 705-713] and higher TP levels [Takebayashi, Y., et al., Eur. J. Cancer 32 (1996) 1227-1232; Miwa, M., et al., Eur. J. Cancer 34 (1998) 1274-1281] than “normal” tissues, a concept underlying tumor-specificity for 5-FU pro-drugs.

Whereas the inventors also detected lower DPD levels in the “tumor” cell than “normal epithelial” cell isolates, the results did not reveal differences between TP and TS mRNA levels in tumor and “normal” cells. This most likely reflects the precision of defining of a “normal” cell population. For interpretation of TP, DPD and TS expresssion, one must carefully control the morphology of the tissue acquired, since non-epithelial cells may significantly influence the results. Thus, when “normal colonic smooth muscle cells” (as opposed to the “normal epithelial cell’ samples) were compared to “tumor” cells, the latter exhibited higher TP and TS mRNA expression, consistent with previous reports [Takebayashi, Y., et al., Eur. J. Cancer 32 (1996) 1227-1232; Miwa, M., et al., Eur. J. Cancer 34 (1998) 1274-1281].

In certain embodiments, the present invention is directed to a quantitative RT-PCR approach for determining 5-FU and/or 5-FU analogs responsiveness in cancer patients. The method employs determination of the TP/DPD ratio and optionally DPD mRNA expression levels in FFPE biopsies from patients. TP/DPD ratios has a predictive value for disease-free survival in adjuvant 5-FU treated colorectal cancer patients.

The following examples, references, and figures are provided to aid the understanding of the present invention, and do not limit the scope of the invention in any way. It is understood that modifications can be made in the procedures set forth without departing from the spirit of the invention.

### DESCRIPTION OF THE FIGURES

FIG. 1 TP, DPD and TS mRNA expression in microdissected tissues. Normal colonic mucosa (n=8) and muscularis propia (n=3), chronic colitis (n=3), colorectal cancer (CRC; n=102) and normal liver (n=1, duplicate). mRNA levels are expressed as relative ratio (mean ±stdev.;).

FIG. 2 TP, DPD and TS mRNA expression in 102 CRC patients. Each symbol reflects one case, with bars and numbers indicating median mRNA levels (relative ratio) for: all cases (squares, n=102), “no CTX” (triangles, n=40) and “CTX” (circles, n=52).

FIG. 3 Association of TP, DPD and TS mRNA expression with tumor histology. Graphical overview of the statistically significant correlations, with columns representing the median level within each sub-group. Y-axis denotates relative mRNA levels or enzyme ratios. For p-values refer to Table 2.

FIGS. 4A-4C Correlation of TP/DPD ratio with overall and disease-free survival. Kaplan-Meier analysis with respect to overall survival for the “no CTX” (n=40) and “CTX” (n=52) groups (A). Neither TP, DPD nor TS mRNA levels were of predictive value for overall survival in 5-FU treated (“CTX”) patients (B, with cut-off=median mRNA expression). However, low DPD mRNA levels ratio were significantly correlated to disease-free survival in 5-FU treated patients (C, with cut-offs as indicated).

FIGS. 5A-5E Correlation of TP/DPD ratio mRNA levels with overall and disease-free survival. Kaplan-Meier analysis with respect to disease free survival and its correlation with the TP/DPD ratio. High TP/DPD ratios were significantly correlated to disease-free survival in 5-FU treated patients (Cut off values: A=0.39, B=3.7, C=5.0, D=6,2, E=8.1).

### EXAMPLES

### Example 1

### Patients and Tissues

Colorectal cancer specimens were obtained from the archive of the Institute of 10 Pathology, Klinikum rechts der Isar, Munich, Germany. Resected primary tumor specimens from a total of 102 patients (Table 1; median clinical follow up=63.5 months, range 8-125 months), were analysed after microdissection of tumor cells.

TABLE 1
Summary of patient characteristics.

| | All | | No CTX¹ | | CTX¹ | |
|---|---|---|---|---|---|---|
| | No. | % | No. | % | No. | % |
| Patients | 102 | | 40 | | 52 | |
| Age: | 62 yrs | | 68 yrs | | 60 yrs | |
| Sex: | | | | | | |
| Male | 67 | 65.7 | 25 | 62.5 | 35 | 67.3 |
| Female | 35 | 34.3 | 15 | 37.5 | 17 | 32.7 |
| Tumor Site: | | | | | | |
| Colon | 65 | 63.7 | 21 | 52.5 | 42 | 80.8 |
| Rectum | 37 | 36.3 | 19 | 47.5 | 10 | 19.2 |
| T Category: | | | | | | |
| T1 | 4 | 3.9 | 3 | 7.5 | 1 | 1.9 |
| T2 | 14 | 13.7 | 9 | 22.5 | 5 | 9.6 |
| T3 | 64 | 62.8 | 26 | 65 | 29 | 55.8 |
| T4 | 20 | 19.6 | 2 | 5 | 17 | 32.7 |
| N Category: | | | | | | |
| N0 | 52 | 51 | 38 | 95 | 4 | 7.7 |
| N1 | 32 | 31.4 | 1 | 2.5 | 31 | 59.6 |
| N2 | 18 | 17.6 | 1 | 2.5 | 17 | 32.7 |
| UICC Stage: | | | | | | |
| I | 12 | 11.8 | 12 | 30 | — | — |
| II | 40 | 39.2 | 26 | 65 | 4 | 7.7 |
| III | 50 | 49 | 2 | 5 | 48 | 92.3 |
| Diff. Grade: | | | | | | |
| 2 | 70 | 68.6 | 31 | 77.5 | 31 | 59.6 |
| 3 | 31 | 30.4 | 9 | 22.5 | 20 | 38.5 |
| 4 | 1 | 1 | — | — | 1 | 1.9 |
| Clinical Data | | | | | | |
| Recurrent disease | 33 | 32.4 | 11 | 27.5 | 22 | 42.3 |
| Disease free | 59 mo. | | 64 mo. | | 56 mo. | |
| survival² | | | | | | |
| Overall survival² | 63.5 mo. | | 65.5 mo. | | 57 mo. | |
| Follow up² | 63.5 mo. | | 65.5 mo. | | 57 mo. | |
| | (8-125) | | (14-125) | | (8-125) | |

¹= 10 cases excluded from statistical analysis due to combination therapies;
²= numbers represent the statistical median (range) of survival in months.

40 patients underwent tumor resection only (“no CTX” group), 52 patients had received adjuvant chemotherapy after resection (“CTX” group) and 10 patients had received a combined adjuvant chemo-/radiotherapy. The adjuvant regimens in the “CTX” group were as follows: 25/52 patients Mayo protocol (6 months of 425 mg/m² 5-FU and 20 mg/m² leucovorin) [O'Connell, M. J., et al., J. Clin. Oncol. 15 (1997) 246-250], 7/52 “Mortel” regimen [450 mg/m² 5-FU and 50 mg/m² levamisol) [Moertel, C. G., et al., N. Engl. J. Med. 322 (1990) 352-358], 5/52 patients Ardalan protocol (24 hours of 2,600 mg/m² 5-FU and 500 mg/m² Leucovorin) [Ardalan, B., et al., J. Clin. Oncol. 9 (1991) 625-630], 14/52 patients modified Ardalan protocol and 1/52 patients SAKK protocol (500 mg/m² 5-FU and 10 mg/m mitomycin C) [SAKK, Lancet 345 (1995) 349-353]. For control purposes, tissue specimens of normal colon, chronic colitis (see below) and normal liver were obtained. All tissues had been formalin-fixed (10% buffered formalin, 24 hrs) and paraffin-embedded (FFPE) according to routine guidelines. Before analysis, histopathology of each specimen was confirmed on hematoxylin stained serial sections.

### Example 2

### Microdissection

Prior to RNA extraction from FFPE-tissues, microtom sections (5 μm) were treated with xylene and graded alcohols under RNase-free conditions. For subsequent microdissection, sections were individually stained with instant hematoxylin (Shandon, Frankfurt, Germany) and tumor cells were dissected under microscopic observation using fine needles (gauge 18). The purity of the dissected tumor cell population was 80-90%.

Control tissues were dissected under equal conditions and included normal colonic mucosa (n=8; epithelial cells) and colonic muscularis propria (n=4; muscle cells), reactive lesions of chronic colitis (n=3; Crohn's disease, ulcerative colitis and diverticulitis of the sigmoid) and normal liver (n=1; all cell populations). For the latter two control groups, duplicate analysis was performed by processing two serial sections of each tissue specimen separately.

### Example 3

### RNA Isolation

In 52 CRC cases, microdissected tissue samples were subjected to RNA isolation as described previously [Lassmann, S., et al., J. Pathol. 198 (2002) 198-206]. In brief, microdissected tumour cells were immediatly placed into Eppendorf tubes, containing digestion buffer (10 mM TrisHCl, 0.1 mM EDTA, 2% SDS and 0.5 mg Proteinase K, all from Sigma, Taufkirchen, Germany). Incubation was overnight (60° C., 350-400 rpm), followed by phenol:choroform extraction and precipitation of nucleic acids in isopropanol. The resulting RNA pellet was further purified by incubation (45 min, 37° C.) with 10 U DNase I (Roche Diagnostics GmbH, Mannheim, Germany), 20 μl DNase buffer (0.4 M TrisHCl, 60 mM MgCl₂, 0.1 M NaCl) and H₂O up to 200 μl. Thereafter RNA was re-extracted by phenol:chloroform extraction, precipitation and resuspension in H₂O. In 50 CRC cases and the control specimens, microdissected tumor or control cells were isolated with the “HighPure RNA Paraffin Kit” (Roche Diagnostics GmbH, Mannheim, Germany) according to the supplied protocol. This method also consists of a Proteinase K digestion step, purification of nucleic acids, a DNase digestion step and re-purification of the RNA. In preliminary experiments similar results were obtained from 3 serial tissue sections of each a normal mucosa and a tumor sample isolated by the two methods (data not shown). RNA was stored at −70° C. until further use.

### Example 4

### Reverse-Transcription and Quantitative Polymerase Chain Reaction (QRT-PCR)

Reverse transcription and quantitative PCR in the LightCycler® system was performed with reagents and kits from Roche Applied Science according to the supplier's instructions. In brief, RNA samples were distributed to four equal aliquots, all receiving the same mix of cDNA reagents and either TP, DPD, TS or reference gene specific primers (Cat. Nos. 3 302 946, 3 302 938, 3 302 954). A positive control RNA (calibrator, from the “LC-mRNA quantification Kits for TP, DPD and TS”) was included in this step. Always one calibrator RNA together with 4 unknown samples was treated as a separate “set” in order to control for quality and reproducibility. Always 3 cDNA “sets” were then analysed together in one run of quantitative PCR, using the “LC-mRNA quantification kits for TP, DPD and TS”. For data analysis, the “Relative Quantification Software” (Roche Diagnostics GmbH, Mannheim, Germany) was applied. This calculates the relative ratio of Cp(enzyme:reference gene)ₛₐₘₚₗₑ to Cp(enzyme:reference gene)_{calibrator}, thereby controling both for sample loading (RNA input) and PCR efficiency due to the constant reference point (calibrator). To ensure accurate quantification, only data of RNA preparations with crossing points of 20 to max. 33 (linear amplification range) were included. The variance of TP, DPD, TS and reference gene mRNA expression (mean ±stdev. of crossing point) was minimal, with 26.62±0.36, 28±0.51, 22±0.23 and 23.19±0.7 for 29 calibrators (accounting for 29×4=116 tissue samples), respectively.

### Example 5

### Statistics

Quantitative parameters were described using mean or median with standard deviation and ranges, respectively. Qualitative parameters were examined by frequency tables. Non-parametric tests were performed (SAS® software; version 8.02), as a deviation from the normal distribution was observed for all markers.

In the group of all 102 cases, TP, DPD and TS mRNA expression as well as the TS/DPD and TP/DPD ratio were correlated to 1) patient age and gender, and 2) primary tumor localisation, TNM classification, UICC stage and differentiation grade. This was done by the Spearman correlation coefficient and the test on zero correlation. For comparison of individual subgroups, the Wilcoxon-test for unpaired samples and the Kruskal-Wallis-test were applied. In order to evaluate the prognostic impact and/or response prediction value of the three enzymes, the 102 cases were divided into the “no CTX” (n=40) and the “CTX” (n=52) group. Patients who had received a combined radio-/chemotherapy (n=10) were excluded. Within the two subgroups separate statistical analysis was performed for correlation of TP, DPD and TS mRNA expression and the TS/DPD and TP/DPD ratios with: incidence of recurrent disease, incidence of death as well as disease-free and overall survival. The survival analysis was achieved by both a Cox-regresssion and a log-rank-test, setting the significance level to 5%.

### Example 6

### Results

TP, DPD and TS mRNA Expression in Normal Colon, Chronic Cholitis and CRC

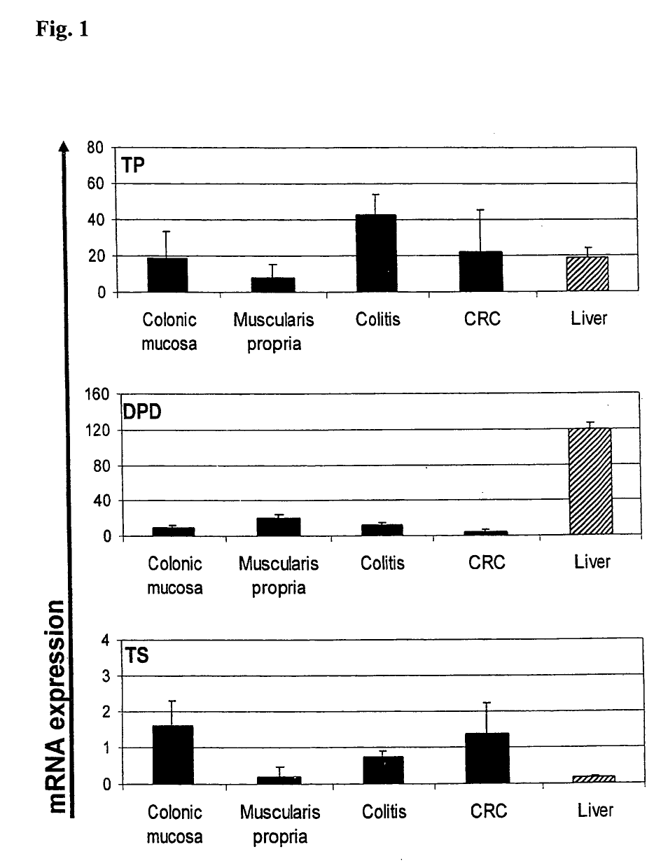

Initially, TP, DPD and TS mRNA levels were examined in a series of microdissected, FFPE control specimens by quantitative RT-PCR using the LightCycler® system. As shown in FIG. 1, mRNA expression for TP, DPD and TS was detected in all of the samples, but mRNA expression levels differed between tissues: High TP mRNA expression was seen in reactive lesions of chronic colitis, followed by moderate levels in normal colonic mucosa (epithelial cells) and normal liver (mixed cell population) and even lower expression in normal colonic muscularis propria (muscle cells). DPD mRNA expression was greatest in normal liver, followed by normal muscularis propria>reactive lesions of chronic colitis≧normal mucosa. TS was highly expressed in normal mucosa>reactive lesions of chronic colitis>normal liver and muscularis propria.

In comparison, the mean TP, DPD and TS mRNA expression levels of colon tumor samples (n=102, see below) revealed a lower DPD mRNA expression in tumor samples when compared to epithelial cells of normal mucosa. In contrast, no significant difference of TP and TS mRNA levels was detected between normal mucosa and tumor tissues.

Screening of TP, DPD and TS mRNA Expression in 102 Patients with Colorectal Cancer

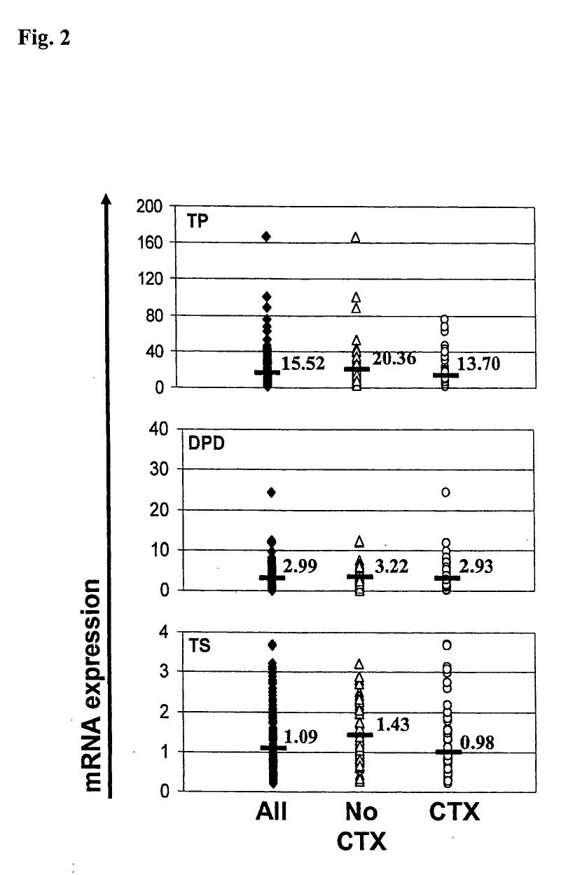

The group of patients examined included 102 cases of CRC of various stages (Table 1, Materials and Methods), either treated with resection alone (40 cases; “no CTX” group), with resection and subsequent 5-FU chemotherapy (52 cases; “CTX” group) or with resection and a combination of radio- and chemotherapy (10 cases). Analysis of the mRNA expression of TP, DPD and TS in all 102 CRC cases showed a wide range of enzyme expression patterns (FIG. 2). Expressed as a relative ratio, the ranges for TP, DPD and TS were 1.52-166.29, 0-24.39 and 0.21-3.71, respectively. Upon division of the cases into groups of “no CTX” and “CTX”, TP, DPD and TS mRNA expression was similar in both groups, except for a trend to lower TP mRNA expression in the “CTX” group. This is reflected by the statistical median of TP, DPD and TS mRNA expression levels (FIG. 2).

Correlation of TP, DPD and TS mRNA Expression to Patient Data and Histology

As summarized in Table 2A, no statistically significant correlation was revealed between TP, DPD or TS mRNA levels or the TS/DPD and TP/DPD ratios with patient age or gender and the location of the primary tumor (colon or rectum).

TABLE 2
Numbers represent p-values of Kruskal-Wallis test for
patient and tumor parameters

| | | | | | TS:DPD |
|---|---|---|---|---|---|
| A | TP | DPD | TS | TP/DPD ratio | ratio |
| Patient | | | | | |
| Age | — | — | — | | — |
| Gender | — | — | — | — | — |
| Tumor | | | | | |
| Location | — | — | — | — | — |
| T Category | 0.03 | — | — | 0.007 | 0.014 |
| N Category | 0.04 | — | — | 0.001 | — |
| UICC Stage | 0.009 | — | — | 0.001 | — |
| Diff. Grade | — | — | 0.0014 | — | 0.033 |

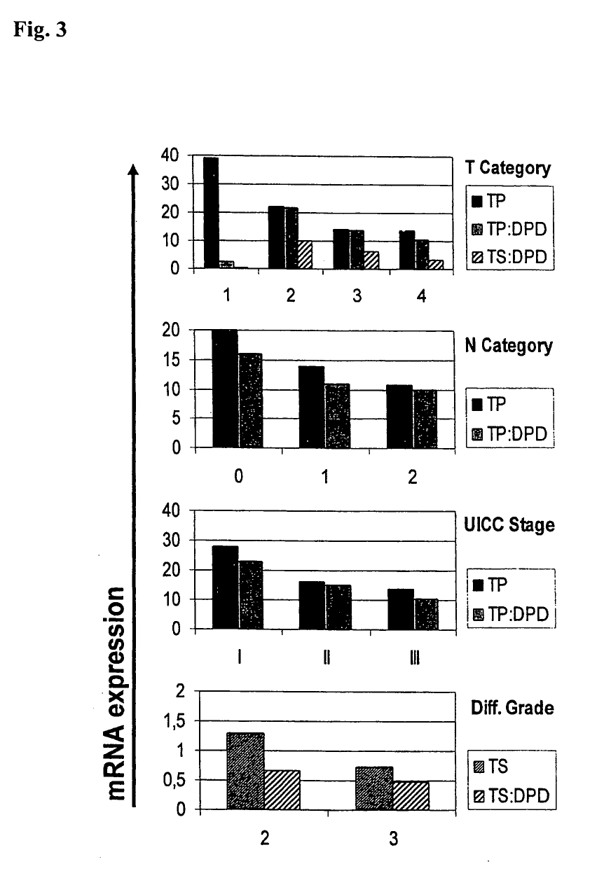

However, significant differences were seen with respect to 1) TP mRNA expression with tumor T (p=0.03) and N (p=0.04) category and UICC stage (p=0.009); 2) TS mRNA expression with differentiation grade (p=0.001), 3) the TS/DPD ratio with tumor T category (p=0.014) and differentiation grade (p=0.033) and 4) the TP/DPD ratio with tumor T (p=0.007) and N (p=0.001) category and UICC stage (p=0.001). As shown in FIG. 3, TP mRNA expression and the TP/DPD ratio significantly decreased with higher tumor T and N categories as well as with higher UICC stages. TS mRNA expression was significantly lower in differentiation grade 3 than grade 2 tumors. Finally, the TS/DPD ratio was lower in tumors with higher T category as well as in differentiation grade 3 than grade 2 tumors.

TP, DPD and TS mRNA Expression in CRC—Correlation to Prognosis

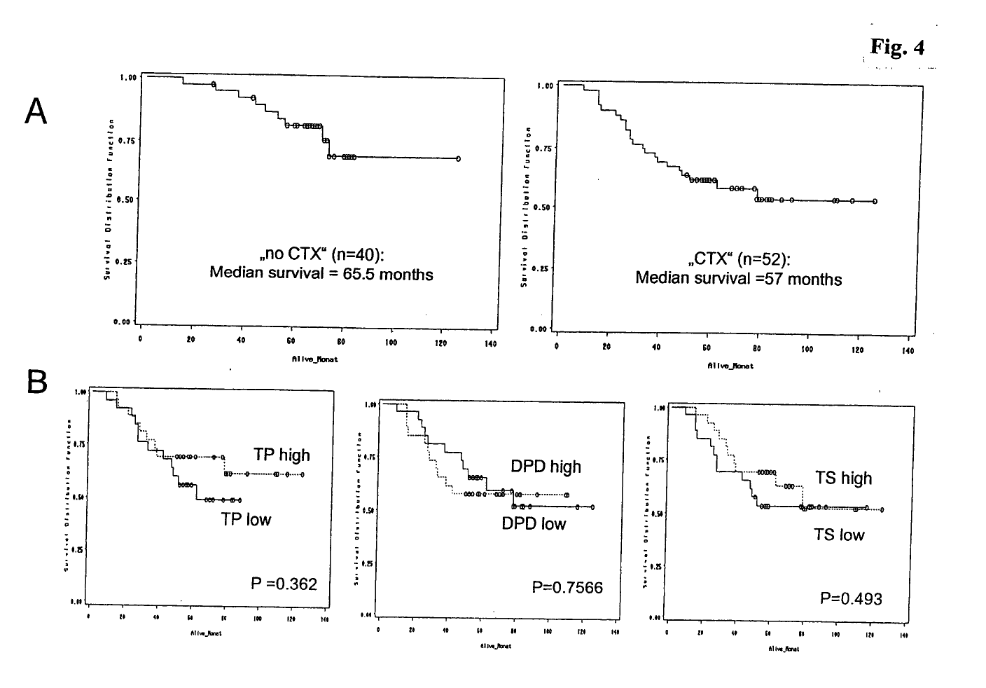

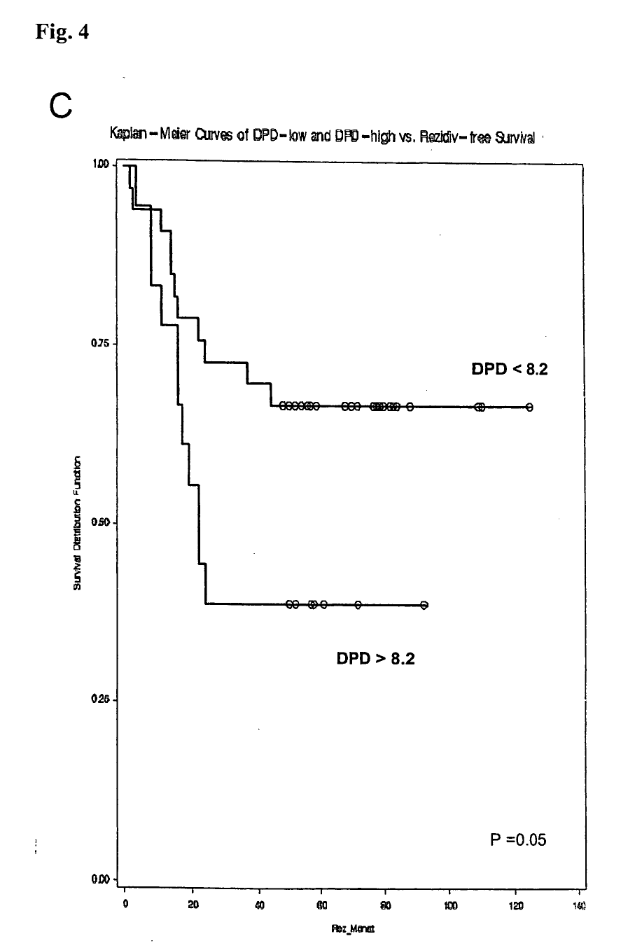

In order to correlate TP, DPD and TS mRNA expression with prognosis, patients who had received adjuvant chemo- and radiotherapy (n=10) were excluded from statistical analysis. Moreover, as patients with lymphnode involvement (“N+”) have in general a poorer prognosis than those who are classified as “NO” [1], the remaining 92 patients were divided into those without adjuvant therapy (n=40; “no CTX”) and those with adjuvant chemotherapy (n=52; “CTX”), as shown in FIG. 4A. Statistical analysis was then performed separately within the two groups with respect to incidence of recurrent disease and death as well as disease-free and overall survival. First, no significant correlation was revealed between either TP, DPD or TS mRNA expression or the TS/DPD and TP/DPD ratios and the incidence of recurrent disease or death (Table 2B).

TABLE 3
Numbers represent p-values of Cox regression
for follow-up parameters

| | | | | | TS:DPD |
|---|---|---|---|---|---|
| B | TP | DPD | TS | TP/DPD ratio | ratio |
| “No CTX” Group | | | | | |
| Recurrent Disease | — | — | — | — | — |
| Overall survival | — | — | — | — | 0.032 |
| “CTX” Group | | | | | |
| Recurrent Disease | — | — | — | — | — |
| Disease-free survival | — | 0.05 | — | 0.002 | — |
| Overall survival | — | — | — | — | — |

Second, none of the enzymes or the TP/DPD ratio had a significant influence on overall survival (Kaplan-Meier analysis). Third, multivariate analysis of TP, DPD and TS mRNA expression and overall survival did not reveal any significant correlation, even though TP and DPD mRNA expression were significantly (p<0.0001) correlated in the “CTX” group. These findings were true for both the “no CTX” or “CTX” group (Table 2).

In contrast, within the “no CTX” group overall survival significantly correlated to the TS/DPD ratio (p=0.032), whereby the risk of death increases with higher TS/DPD ratios.

TP, DPD and TS mRNA Expression—Markers for Response Prediction

To evaluate whether TP, DPD and/or TS mRNA levels or the ratio of TP/DPD or TS/DPD can predict the clinical response to 5-FU chemotherapy, detailed statistical analysis was performed for patients within the “CTX” group. Upon sub-division of the “CTX” patients into those with “low” and “high” TP, DPD and TS mRNA levels (cut off=median, as indicated in FIG. 2) and subsequent Kaplan Meier analysis, no correlation was found with respect to overall survival (FIG. 4B). Similarly, neither low or high TP/DPD or TS/DPD ratios did predict for overall survival in the two “CTX” sub-groups (not shown).

However, significant correlations were seen for DPD mRNA levels and the TP/DPD ratio with respect to disease-free survival (FIG. 4C). Thus, using a cut-off mRNA level of 8.2, low DPD mRNA expression was correlated to disease-free survival with p=0.05.

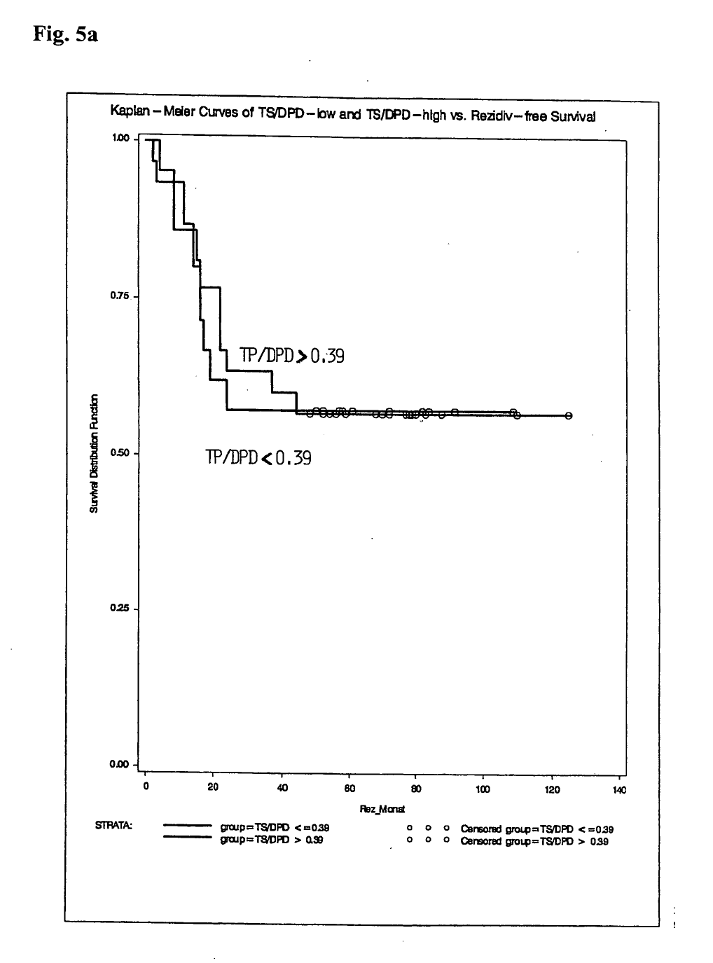

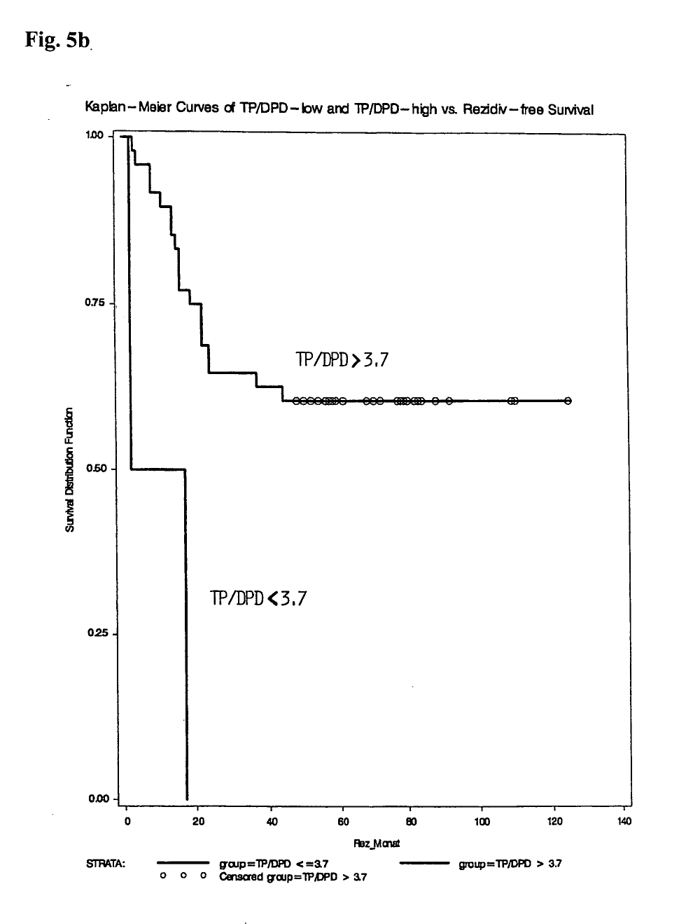

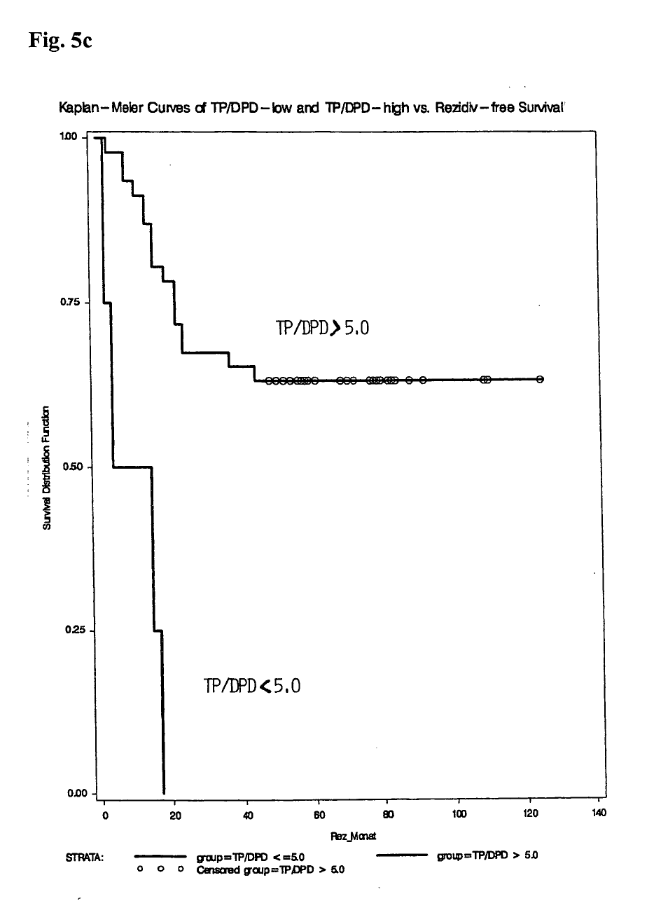

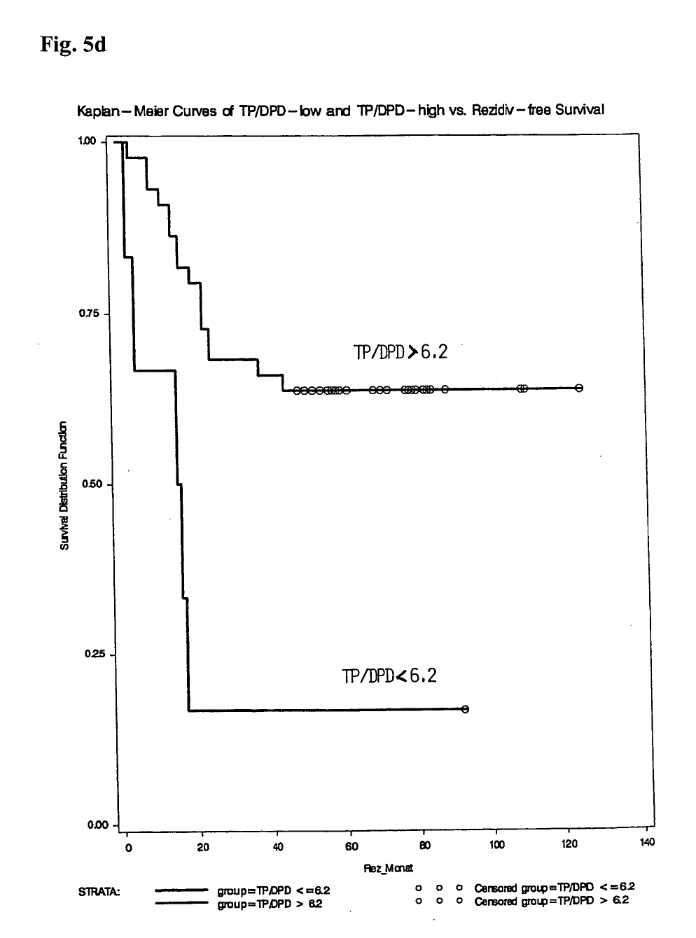

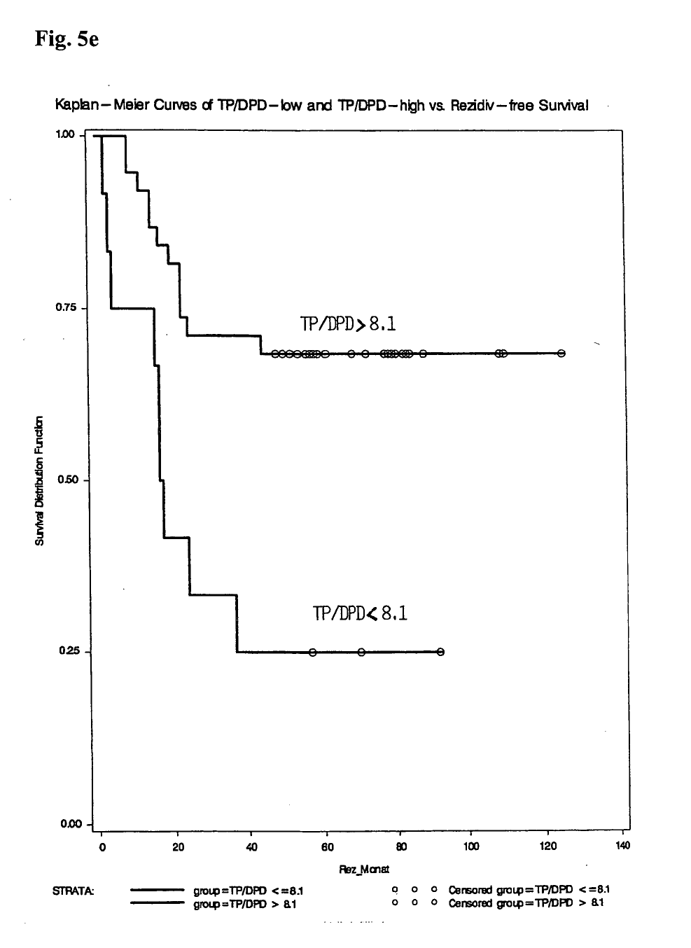

Moreover, a positive correlation between disease free survival and TP/DPD ratio has been identified. As it is shown in FIG. 5, using cut-off values of 3.7 (FIG. 5b), 5.0 (FIG. 5c), 6.2 (FIG. 5d), and 8.1 (FIG. 5e), a high TP/DPD ratio was significantly correlated to disease-free survival with p=0.002.

List of References

Allegra, C. J., et al., J. Clin. Oncol. 20 (2002) 1735-1743
Araki, Y., et al., Kurume Med. J. 48 (2001) 93-98
Ardalan, B., et al., J. Clin. Oncol. 9 (1991) 625-630
Backus, H. H., et al., J. Clin. Pathol. 55 (2002) 206-211
Bathe, O. F., et al., Cancer J. Sci. Am. 5 (1999) 34-40
Copur, S., et al., Biochem. Pharmacol. 49 (1995) 1419-1426
Diasio, R. B., and Johnson, M. R., Pharmacology 61 (2000) 199-203
Edler, D., et al., J. Clin. Oncol. 20 (2002) 1721-1728
Findlay, M. P., et al., Br. J. Cancer 75 (1997) 903-909
Fox, S. B., et al., J. Pathol. 176 (1995) 183-190
Furukawa, T., et al., Nature 356 (1992) 668
Griffiths, L, and Stratford, I. J., Br. J. Cancer 76 (1997) 689-693
Guimbaud, R., et al., Cancer Chemother. Pharmacol. 45 (2000) 477-482
lacopetta, B., et al., Br. J. Cancer 85 (2001) 827-830
Iqbal, S., and Lenz, H. J., Curr. Oncol. Rep. 3 (2001) 102-108
Ishikawa, T., et al., Cancer Res. 58 (1998) 685-690
Johnson, M. R., et al., Clin. Cancer Res. 5 (1999) 2006-2011
Johnston, P. G., et al., Cancer Res. 55 (1995) 1407-1412
Johnston, S. J., et al., Clin. Cancer Res. 5 (1999) 2566-2570
Kawakami, K., et al., Clin. Cancer Res. 7 (2001) 4096-4101
Kornmann, M., et al., J. Gastrointest. Surg. 6 (2002) 331-337
Kumar, S. K., and Goldberg, R. M., Curr. Oncol. Rep. 3 (2001) 94-101
Lassmann, S., et al., J. Pathol. 198 (2002) 198-206
Marsh, S., et al., Int. J. Oncol. 19 (2001) 383-386
Metzger, R., et al., Clin. Cancer Res. 4 (1998) 2371-2376
Miwa, M., et al., Eur. J. Cancer 34 (1998) 1274-1281
Miyamoto, S., et al., Int. J. Oncol. 18 (2001) 705-713
Moertel, C. G., et al., N. Engl. J. Med. 322 (1990) 352-358
Mori, K., et al., Int. J. Oncol. 17 (2000) 33-38
O'Connell, M. J., et al., J. Clin. Oncol. 15 (1997) 246-250
Paradiso, A., et al., Br. J. Cancer 82 (2000) 560-567
Pestalozzi, B. C., et al., Br. J. Cancer 71 (1995) 1151-1157
Pestalozzi, B. C., et al., Br. J. Cancer 71 (1995) 1151-1157
Ragnhammar, P., et al., Acta Oncol. 40 (2001) 282-308
Raida, M., et al., Clin. Cancer Res. 7 (2001) 2832-2839
SAKK, Lancet 345 (1995) 349-353
Salonga, D., et al., Clin. Cancer Res. 6 (2000) 1322-1327
Sanguedolce, R., et al., Anticancer Res. 18 (1998) 1515-1520
Sumizawa, T., et al., J. Biochem. 114 (1993) 9-14
Takebayashi, Y., et al., Eur. J. Cancer 32 (1996) 1227-1232
Takebayashi, Y., et al., J. Natl. Cancer Inst. 88 (1996) 1110-1117
Takenoue, T., et al., Ann. Surg. Oncol. 7 (2000) 193-198
Tanaka-Nozaki, M., et al., Clin. Cancer Res. 7 (2001) 2783-2787
Uetake, H., et al., Clin. Cancer Res. 5 (1999) 2836-2839
van Kuilenburg, A. B., et al., Clin. Cancer Res. 6 (2000) 4705-4712
Wang, W., et al., Cancer Res. 61 (2001) 5505-5510
Wei, X., et al., J. Clin. Invest. 98 (1996) 610-615
WO 02/44423

1. A method for determining whether a patient suffering from cancer will be responsive to a treatment with a 5-Fluoro-Uracil and/or 5-FU analogs comprising:
a) determinating a value of a level of Thymidine Phosphorylase mRNA expression in a clinical sample;
b) determinating a value of a level of Dihydropyrimindine Dehydrogenase mRNA expression in the clinical sample;
c) determinating a ratio of the value obtained in step a) and the value obtained in step b); and
d) determinating whether the ratio obtained in step c) exceeds a predetermined cut off value.

2. The method according to claim 1, wherein the cut off value is 3.

3. The method according to claim 1, wherein the cut off value is 3.7.

4. The method according to claim 3, wherein said cut off value is not higher than 10.

5. The method according to claim 3, wherein said cut off value is not higher than 8.2.

6. The method according to claim 1, further comprising the steps of:
a) determining an absolute or relative mRNA expression level of Dihydropyrimidine Dehydrogenase; and
b) determining an indication for responsiveness to 5 Fluoro-Uracil and/or 5-FU analogs if the expression level is below a cut off value.

7. The method according to claim 1, wherein the cancer is colorectal cancer.

8. The method according to claim 6, wherein levels of mRNA expression of Thymidine Phosphorylase and/or Dihydropyrimindine Dehydrogenase are determined by Reverse Transciptase PCR.

9. The method according to claim 8, wherein a sequence specific Thymidine Phosphorylase primer and/or a sequence specific Dihydropyrimindine Dehydrogenase primer is elongated during a cDNA synthesis step.
